(19) United States
(12) Patent Application Publication
(10) Pub. No.: US 2018/0353189 A1
(43) Pub. Date: Dec. 13, 2018

(54) TOURNIQUET

(71) Applicant: The Henry M. Jackson Foundation for the Advancement of Military Medicine, Inc., Bethesda, MD (US)

(72) Inventors: Craig Goolsby, Chevy Chase, MD (US); Elizabeth P. Chen, Gaithersburg, MD (US); Elizabeth N. Weissbrod, Silver Spring, MD (US); Andrew J. Branting, Rockville, MD (US)

(21) Appl. No.: 15/562,355

(22) PCT Filed: Apr. 22, 2016
(86) PCT No.: PCT/US16/29004

§ 371 Date: Sep. 27, 2017

Related U.S. Application Data
(60) Provisional application No. 62/152,174, filed on Apr. 24, 2015.

(51) Int. Cl. A61B 17/132; A44B 11/18; G09B 19/00; G09B 5/04; A61B 90/92

(52) U.S. Cl. CPC A61B 17/1322; A44B 11/18; A61B 2017/00115; G09B 5/04; A61B 90/92; G09B 19/00

(57) ABSTRACT

A buckle includes a frame, an intermediate bar integrally connected at two opposite sides of the frame and defining two ports in the frame; and a barrier bar integrally connected at the two opposite sides of the frame and spaced apart from the intermediate bar forming a third port between the intermediate bar and the barrier bar. Optionally, the buckle may be a part of a tourniquet that further includes a retainer, including a first portion having a catch for securing a windlass rod, the catch having at least one top portion, where a surface of the top portion includes a first coupling member; and a second portion having a third strap, where the third strap includes a second coupling member.

### RELATED APPLICATIONS

This application claims priority to U.S. Provisional Patent Application No. 62/152,174 entitled “TOURNIQUET AND METHOD OF USE” filed on Apr. 24, 2015, the entire contents of which are incorporated herein by reference in its entirety.

### BACKGROUND

### 1. Field of the Disclosure

The disclosure relates generally to tourniquets. More particularly, aspects of the disclosure include tourniquets that are easy to apply, and/or tourniquet timers that provide visual indicators.

### 2. Related Art

As known in the art, and as variously described in various of the below-identified references, a tourniquet is a device that is designed to be applied to a limb for the purpose of constricting blood flow to that limb by applying pressure in order to limit the effects of extreme blood loss.

Tourniquets are typically used in the temporary treatment of extremity injuries, i.e. damage to the body's arms and legs. The severity of an extremity injury depends on the location and path of the injury. Critical conditions are commonly associated with compromised vascular components of the particular extremity. Vascular injuries to the extremities become critical when life threatening blood loss is possible.

When a major artery is severed, either by injury or surgical intervention, controlling blood loss becomes vital. In severe cases, where potential blood loss is considered life-threatening, a tourniquet may be applied proximal to the vascular disruption to manage blood loss. Because of its rapid blood flow occlusion capabilities, the tourniquet is commonly considered a life-saving device in emergency situations.

Even though civilian extremity injuries are common, extremity injuries occurring in the battlefield are more frequent and can present unique challenges. Therefore, emergency tourniquets may be specialized for use on the battlefield. In general, the goal of such tourniquets is to extend the survival time of a casualty until the patient can reach additional medical aid.

Tourniquets have proven to dramatically reduce battlefield deaths. Unfortunately, many of those combat-type injuries seen overseas are now occurring off the battlefield at an increasing rate as seen with the Boston Marathon bombings, and Charlie Hebdo shootings. In these mass casualty situations, medical personnel are limited and these injuries are time sensitive (for example, hemorrhagic shock can set in within minutes). However, it is difficult for layperson bystanders to be able to successfully apply tourniquets that are designed for experienced medical personnel.

### BRIEF SUMMARY

As described herein, one or more embodiments provide for a slide buckle, comprising a frame; an intermediate bar integrally connected at two opposite sides of the frame and defining two ports in the frame; and a barrier bar integrally connected at the two opposite sides of the frame and spaced apart from the intermediate bar forming a third port between the intermediate bar and the barrier bar.

According to one or more embodiments, the bar includes a plurality of protrusions extending into at least one of the two slots, wherein the plurality of protrusions prevent slipping of straps threaded in the respective two ports.

According to one or more embodiments, a tourniquet, is disclosed comprising the slide buckle, a base member comprising a base member frame; a base member intermediate bar defining two ports in the base member frame, wherein a portion of a second strap is threaded through one of the two ports in the base member frame and another portion of the second strap is threaded through one of the two ports in the frame of the slide buckle and the third port of the slide buckle; and an extension portion of the base member; and the first strap; wherein the first strap has a first end connected to the extension portion of the base member.

According to one or more embodiments, the tourniquet further comprises a retainer, comprising: a first portion having a catch for securing a windlass rod, the catch having at least one top portion, where a surface of the top portion includes a first coupling member; and a second portion having a third strap, where the third strap includes a second coupling member.

According to some embodiments, the first and second coupling members are made of material including one of a hook and loop structure, magnets, and buttons.

According to some embodiments, the tourniquet includes at least one area including a set of instructions for applying the tourniquet.

According to some embodiments, the third strap includes a timing device.

According to some embodiments, the timing device includes a reset feature to reset a timing of the timing device, the reset feature being configured to be depressed into a surface of the timing device.

According to some embodiments, the windlass rod is operatively associated with a fourth strap and configured to engage with the catch of the retainer.

According to some embodiments, the windlass rod includes a central portion and a peripheral portion and wherein the central portion is of a first color.

According to some embodiments, the slide buckle is a second color and the base member, the retainer, and the catch are a third color and each of the first color, the second color, and the third color is different.

According to some embodiments, the peripheral portion of the windlass is the same color as the base member, the retainer, and the clip.

According to some embodiments, the tourniquet has imprinted thereon a set of instructions, and at least one instruction of the set of instructions relates to operation of the slide buckle, the central portion or peripheral portion of the windlass rod, or the retainer, and the at least one instruction of the set of instructions is provided in the same color of the slide buckle, the central or peripheral portion of the windlass rod, or the retainer that the at least one instruction of the set instructions corresponds to.

According to some embodiments, the tourniquet further comprises an audio section having an activation mechanism; and a speaker, wherein the audio section is configured to play pre-recorded instructions for using the tourniquet.

According to some embodiments, the tourniquet further comprises a physiological tracker for collecting and storing at least one biomarker.

According to some embodiments, the tourniquet further comprises a timer section comprising an activation mechanism; and a display.

According to some embodiments, the timer activation mechanism is automatically activated upon application of the tourniquet to an injured person.

Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification; illustrate embodiments of the disclosure and together with the detailed description serve to explain the principles of the disclosure. No attempt is made to show structural details of the disclosure in more detail than may be necessary for a fundamental understanding of the disclosure and various ways in which it may be practiced.

FIG. 1 is a perspective view of an embodiment of the present disclosure applied to a person's right leg (as shown in dashed lines).

FIG. 2 is a plan view of the device shown in FIG. 1, where the device is stretched out along its longitudinal axis.

FIG. 3 is a side elevation view of the device shown in FIG. 1, where the device is shown prior to tightening the device using the windlass.

FIG. 4 is a cross sectional view of the device shown in FIG. 2 with the windlass in an unwound position.

FIG. 5 is the same cross section view of the device as shown in FIG. 4, but with the outer sleeve looped through the buckle and the windlass partially rotated.

FIG. 6 is a cross sectional view of a portion of the device with the windlass in a wound position.

FIG. 7 is a plan view of the buckle end of the device with the outer sleeve looped through the buckle and the windlass in an unwound position.

FIG. 8 is a plan view of the buckle end of the device with the outer sleeve looped through the buckle and the windlass in a wound position.

FIG. 9 is a perspective view of a buckle of the device according to one embodiment of the present disclosure.

FIG. 10 is a side elevation view of the buckle of FIG. 9 according to one embodiment of the present disclosure.

FIG. 11 is a plan view of the buckle of FIG. 9 according to one embodiment of the present disclosure.

FIG. 12 is a front view of the buckle of FIG. 9 according to one embodiment of the present disclosure.

FIG. 13 is a perspective view of the buckle of FIG. 9 showing the first end of the outer sleeve being looped through the first and second ports of the buckle according to one embodiment of the present disclosure.

FIG. 14 is a cross sectional view of a portion of the buckle of FIG. 9 illustrating the interaction of the first and second tooth sets with the outer sleeve.

FIG. 15 is a perspective view of a buckle of the device according to another embodiment of the present disclosure with a second tooth set inverted relative to a first tooth set.

FIG. 16 is a cross sectional view of a portion of the buckle of FIG. 15 illustrating the interaction of the first and second tooth sets with the outer sleeve.

FIG. 17 is a perspective view of a buckle of the device according to another embodiment of the present disclosure with only a single tooth set.

FIG. 18 is a perspective view of a buckle of the device according to another embodiment of the present disclosure with an elevated intermediate bar.

FIG. 19 is a side elevation view of the buckle of FIG. 18 showing the first end of the outer sleeve being looped through the first and second ports of the buckle according to another embodiment of the present disclosure.

FIG. 20 is a perspective view of a buckle of the device according to another embodiment of the present disclosure with an elevated intermediate bar and first and second tooth sets.

FIG. 21 is a perspective view of a buckle of the device according to another embodiment of the present disclosure with an elevated intermediate bar and a second tooth set inverted relative to a first tooth set.

FIG. 22 is a perspective view of a buckle of the device according to another embodiment of the present disclosure with an elevated intermediate bar and only a single tooth set.

FIG. 23 is a top and bottom view of a tourniquet in accordance with one or more embodiments of the present disclosure.

FIG. 24 is an example set of printed instructions, in accordance with one or more embodiments of the present disclosure.

FIGS. 25-30 depict a front and back view of a tourniquet in according with one or more embodiments of the present disclosure.

FIG. 31 depicts an example instruction card in accordance with one or more embodiments of the present disclosure.

The drawings are not necessarily to scale, and may, in part, include exaggerated dimensions for clarity.

### DETAILED DESCRIPTION

As discussed above, there is an increase in casualties that occur off the battlefield where medical personnel may not be available to assist in stabilizing the injured until medical personnel may intervene.

As discussed herein, a tourniquet is disclosed that is designed in such a way that a layperson, i.e., not trained medical personnel, may be able to properly apply the tourniquet to an injured person.

The present disclosure comprises a tourniquet including a slide buckle designed such that the tourniquet can be easily applied to an injured person by the user.

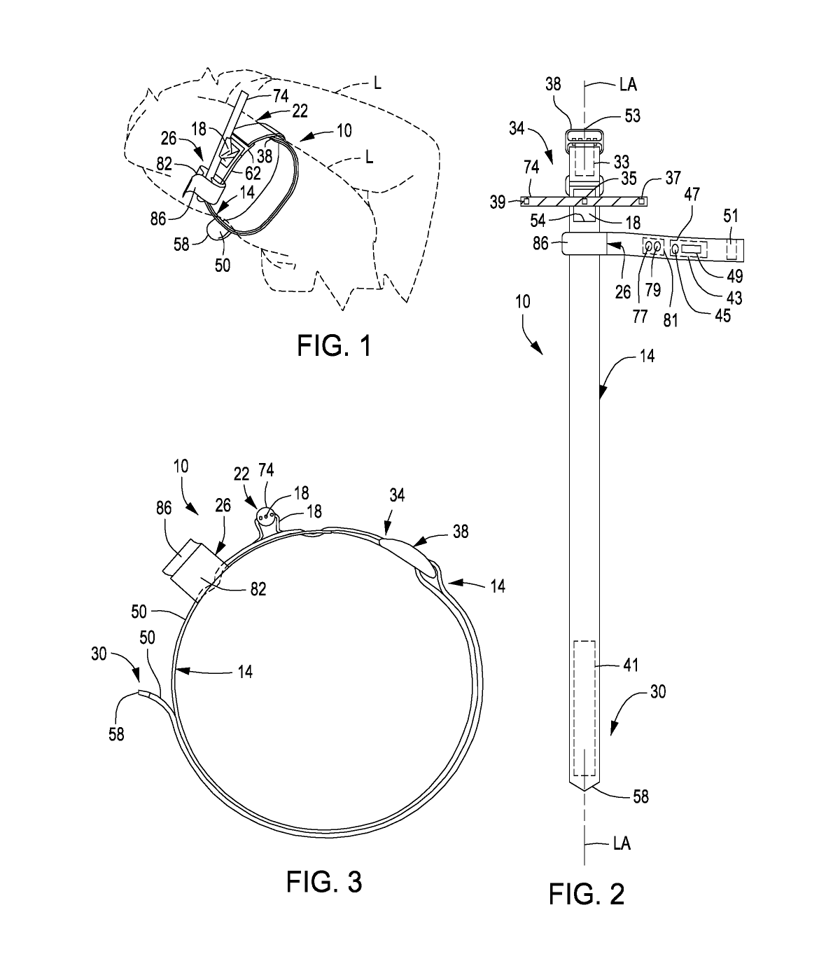

Referring now to FIG. 1, a tourniquet 10 in accordance with embodiments of the present disclosure is shown. The tourniquet 10 comprises a first elongated member or an outer sleeve 14, a second elongated member, inner tightening member or inner strap 18, a tightening mechanism 22 and a securing mechanism 26. As shown in FIG. 1, the tourniquet 10 can be applied to an appendage, as for example, leg L, and then tightened to restrict the flow of blood to the leg L.

Referring now to FIG. 2, the tourniquet 10 is shown prior to use, or in a stretched-out orientation. The outer sleeve 14 comprises a longitudinally extensive material having a first end 30 and a second end 34. In accordance with embodiments of the present disclosure, the second end 34 includes a restraining mechanism comprising a buckle 38. When the tourniquet 10 is applied to a limb, such as leg L shown in FIG. 1, the first end 30 is looped through the buckle, for example, a slide buckle, 38 and pulled tight around the appendage, thus providing a means for circumferentially surrounding or encircling the limb. FIG. 3 depicts the tourniquet 10 after the first end 30 has been looped through the buckle 38.

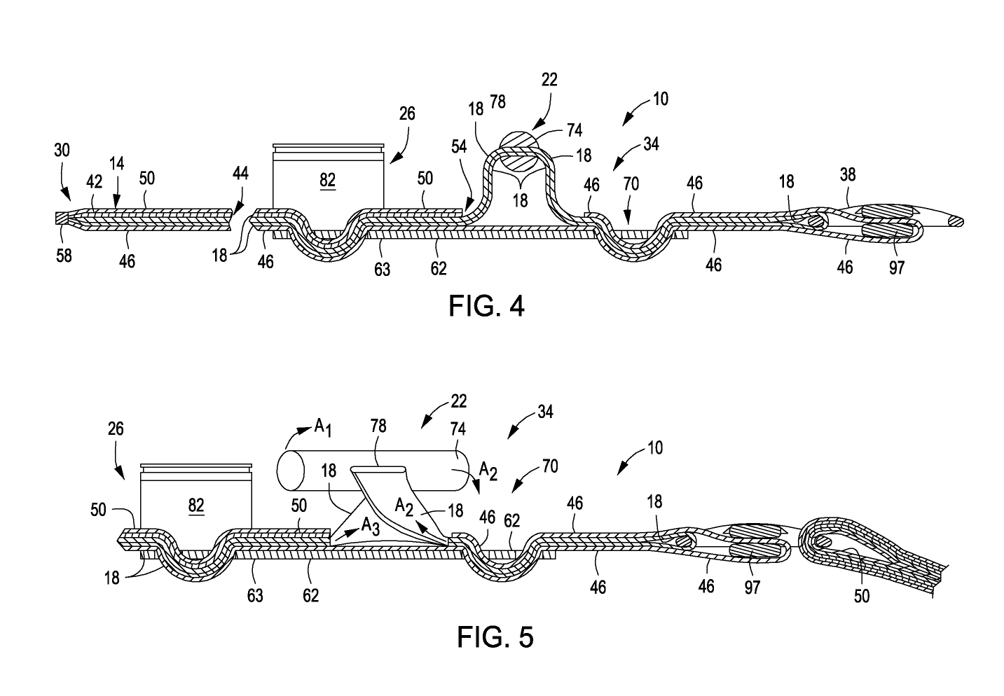

Referring now to FIG. 4, in accordance with embodiments of the present disclosure, the outer sleeve 14 may be formed of two panels comprising an upper or first panel 42 and a lower or second panel 46. The edges of the panels 42 and 46 are connected, as for example, by sewing, gluing, stapling, clamping, or heat/ultra-sound (sonic) welding, or combinations thereof. Outer sleeve 14 includes a pocket, interior area or inner space 44 between the panels 42 and 46. The first panel 42 comprises an outer surface 50 that preferably includes hook and loop structures. More preferably, the outer surface 50 comprises both hook structures and loop structures along substantially the entire length of the outer sleeve 14 between the first end 30 and an opening 54 where the inner strap 18 is exposed between the first panel 42 and second panel 46 of the outer sleeve 14. Thus, when the first end 30 of the outer sleeve 14 is looped through the buckle 38 at the frame 99 of the buckle 38, the outer surface 50 may be applied to itself, thereby securing the position of the outer sleeve 14. By way of example and not limitation, the first panel 42 may comprise a length of OMNI-TAPE® (Velcro Industries B.V., Amsterdam, Netherlands), wherein the fastening surface comprises both hook and loop structures on the outer surface 50 as depicted in FIG. 4. The use of a combination of both hook and loop structures on the outer surface 50 of the outer sleeve 14 provides the advantage of the tourniquet being quickly adjustable when in use to accommodate a variety of size appendages, as for example, from a person's thigh to a person's forearm.

In use, to size the tourniquet to the appendage, the user simply wraps the tourniquet around the subject appendage, loops the first end 30 of the outer sleeve 14 through the buckle 38, pulls the tourniquet reasonably tight, and then presses the outer surface 50 together detachably interlocking first and second portions of the outer surface 50 together to interlock the hook and loop structures of the outer surface 50 within the region where the outer surface 50 overlaps beyond the buckle 38. As those skilled in the art will appreciate, although not preferred, the outer surface 50 of the outer sleeve 14 may be fitted with standard hook fasteners to match-up with corresponding standard loop fasteners; however, although within the scope of the present disclosure, the ability of a single tourniquet so modified to accommodate various size appendages would be limited. Nonetheless, such an issue could be addressed by manufacturing tourniquets of different sizes and/or providing tourniquets having different portions of the outer surface fitted with various lengths of hook material to match-up with corresponding portions of loop material. Alternatively, other means of fastening the overlapping portion of the outer sleeve may be provided, such as buttons, snaps, transverse straps etc., and such variations and modifications are within the scope of the present disclosure.

It is further noted that although the outer sleeve 14 is preferably formed of an upper or first panel 42 and a lower or second panel 46, the outer sleeve 14 may be formed of a single piece of material, as by way of example and not limitation, a piece of material that is folded over and seamed, thereby forming a pocket or inner space 44.

Referring still to FIG. 4, the inner strap 18 is shown between the first panel 42 and the second panel 46 of the outer sleeve 14. In accordance with at least one embodiment of the present disclosure, the inner strap 18 comprises a length of nylon binding strap (also known as nylon binding tape) that extends from first end 30 of the outer sleeve 14 to the buckle 38 and returns to the first end 30 such that the inner strap 18 comprises a loop. Although a substantially non-elastic nylon binding strap type of material is preferred for use as the inner strap 18, other elongated types of materials may be used, such as a section of rope, belt, tubing, hose, band, or combinations thereof, where such structures thereby form a means for compressing a body part. The ends of the inner strap 18 are preferably anchored only at the tip 58 of the first end 30 of outer sleeve 14, as for example, by sewing, gluing, stapling, clamping, or heat/ultra-sound (sonic) welding, or combinations thereof. Thus, the inner strap 18 can slide within the interior space 44 of the outer sleeve 14. Accordingly, the inner strap 18 comprises a material that has frictional characteristics allowing it to slide within the interior space 44 of the outer sleeve 14 when a tensile force is applied to the inner strap 18. Although not required, depending upon the types of materials used to form the outer sleeve 14 and the inner strap 18, the interior space 44 of the outer sleeve 14 may optionally include a substance, such as a powder or other lubricant, to assist with the frictional characteristics between the surfaces of the inner strap 18 and the interior space 44 of the outer sleeve 14.

In accordance with embodiments of the present disclosure, the tourniquet may comprise an inner strap 18 that extends through and end or a slit (not shown) at the first end 30, such as a slit in the upper or first panel 42 of the outer sleeve 14. The inner strap 18 may then be anchored at or proximate to the distal end of the lower or second panel 46. Alternatively, the slit (not shown) may be in the second panel 46 and the inner strap 18 anchored at or proximate to the distal end of the first panel 42.

In accordance with embodiments of the present disclosure, the tourniquet may be configured such that a single layer (i.e., not a loop) of material is used to form the inner strap 18. Here, a first end of the inner strap 18 is anchored at or near the tip 58 of the first end 30 of the outer sleeve 14, and a second end of the inner strap 18 is anchored at or near the buckle 38, with the middle portion not anchored to the outer sleeve 14, and thereby able to slide within the outer sleeve 14. The tensioning mechanism 22 can be used to tighten the inner strap 18, such as by winding the windlass 74 to develop a tension force in the inner strap 18.

Referring still to FIG. 4, in accordance with embodiments of the present disclosure, the tourniquet 10 preferably includes a base member 62 having an extension portion 63. As by way of example and not limitation, the base member 62 may be formed of a KYDEX® (Kleerdex Company, LLC, Mount Laurel, N.J.) thermoplastic or moldable (as for example, injection molded) plastic type of material. A first end 66 of base member 62 preferably includes a securing mechanism 26, as will be discussed below. The second panel 46 of the outer sleeve 14 extends over at least a portion of the base member 62, passes through a means for looping, such as a barrier bar 97 in buckle 38, and folds back to a second end 70 of the base member 62. The edges of the second panel 46 between the buckle 38 and the second end 70 of the base member 62 are preferably connected, as for example, by sewing, gluing, stapling, clamping, or heat/ultra-sound (sonic) welding, thereby securing the second end 34 of the outer sleeve 14 to the buckle 38.

Referring still to FIG. 4, in accordance with embodiments of the present disclosure, the inner strap 18 emerges from the outer sleeve 14 at opening 54 where it is connected to the tightening mechanism 22. For the embodiment shown in FIG. 4, the tightening mechanism 22 comprises a windlass 74 that is shown in an unwound position. The windlass 74 preferably is comprised of a plastic material; however other types of materials are within the scope of the disclosure. In accordance with embodiments of the present disclosure, the inner strap 18 passes through a slot or aperture 78 in the windlass 74, and as described above, the inner strap 18 extends to and around the buckle 38.

By providing for the inner strap 46 to be looped through the barrier bar 97 in buckle 38, when the first end 30 of the strap 14 is prevented from being improperly looped through a first port 126 of the buckle 38, thereby ensuring properly application of the tourniquet.

Referring to FIG. 7, a plan view of the second end 34 of the outer sleeve 14 is shown. Here, the outer sleeve 14 has been looped through buckle 38; however, the tension mechanism 22, comprising a windlass 74, as will be described below, has not been wound to tighten the inner strap 18.

Referring now to FIG. 8, a plan view of the second end 34 of the outer sleeve is shown. Here, the outer sleeve 14 has been looped through buckle 38 and the windlass 74 has been partially wound, thereby applying a tensile force to the inner strap 18. Since the end of the inner strap 18 is secured to the tip 58 of the outer sleeve 14, when the windlass 74 is rotated, the inner strap 18 slides within the outer sleeve 14, essentially scrunching the outer sleeve 14 relative to the inner strap 18 as the inner strap 18 is increasingly tightened. The tightened inner strap 18 provides a substantially even radial compressive pressure to the limb to which the tourniquet 10 is being applied.

Referring now to FIG. 5, a cross sectional view of the tourniquet 10 is shown, including the second end 34 of tourniquet 10 with the windlass 74 in a partially wound position. More particularly, in use, after the first end 30 of the outer sleeve 14 is passed through the buckle 38 and secured around an appendage or limb, such as leg L shown in FIG. 1, the windlass 74 is rotated, such as in the direction of arrows A1 and A2, to apply a tensile force to at least a portion of the inner strap 18. Since the inner strap 18 is secured to the tip 58 of first end 30 of the outer sleeve 14, the inner strap 18 slides in the direction of arrows A3 and A4 within the outer sleeve 14 as the windlass 74 is rotated, thereby pulling the inner strap and providing a circumferentially applied compression force to the appendage. The tensile force is primarily developed in the portion of the inner strap 18 between the buckle 38 and the windlass 74, with typically a lesser amount of tension developed in the overlapping portion of the inner strap 18 between the buckle 38 and the tip 58, because when the inner strap 18 bends around the buckle 58 after being applied to an arm or leg, the bend tends to prevent the slippage of the inner strap 18 in the overlapped portion. After the windlass 74 is tightened, the tourniquet restricts the blood flow in the appendage. Accordingly, the tourniquet 10 of the present disclosure offers the advantage of an unlimited number of possible twists. More particularly, many tourniquets of the prior art are limited to a set number to twists by their windlass, thus limiting the amount of possible compression. As a result, such tourniquets of the prior art are venous tourniquets and are not suitable for arterial occlusion. However, the combination of the outer sleeve 14, inner strap 18 and tightening mechanism 22 of the present disclosure overcome this prior art limitation.

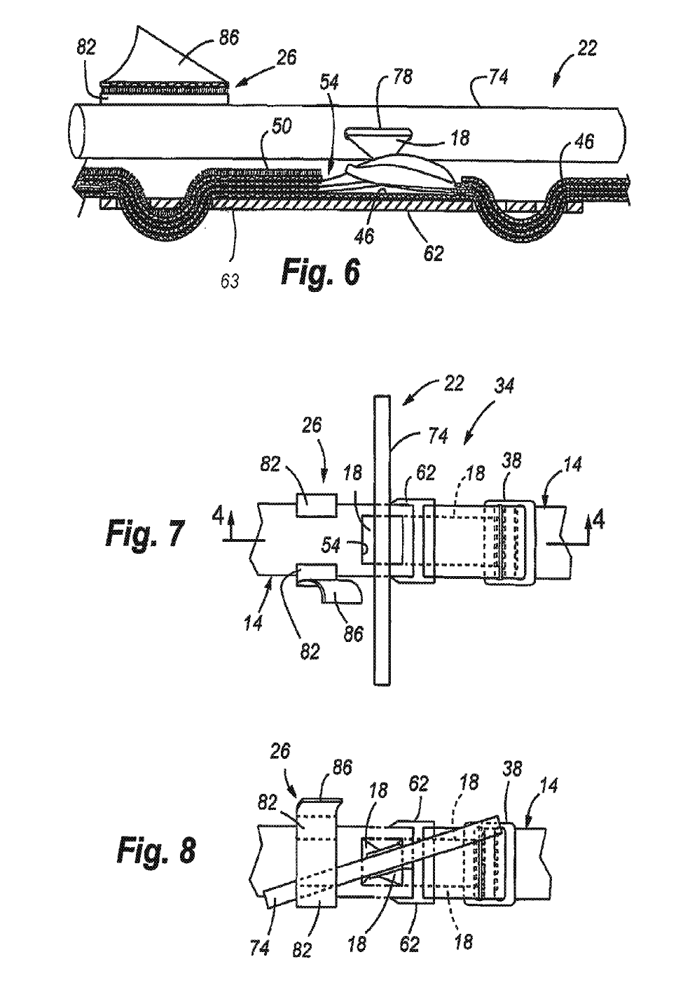

Referring now to FIG. 6, a cross sectional view of the second end 34 of tourniquet 10 is shown with the windlass 74 in a wound position. In accordance with embodiments of the present disclosure, after the windlass 74 has been sufficiently tightened to restrict the arterial blood flow in the appendage, the windlass 74 may be secured using securing mechanism 26. The securing mechanism 26 provides a means for securing or preventing the windlass 74 from unwinding. Thus, the securing mechanism 26 maintains the wound position of the windlass 74, and thereby maintains the tension in the inner strap 18.

In accordance with embodiments of the present disclosure, and as best seen in FIGS. 1, 7, and 8, the securing mechanism 26 preferably comprises a pair of opposing hooked catches 82 set substantially transverse to the longitudinal axis L-L of the tourniquet 10. More particularly, the hooked catches 82 are preferably sized to cup or hold the windlass, or a portion thereof, and prevent it from unwinding. Accordingly, the hooked catches 82 are sufficiently stiff to provide adequate resistance against the tensile force within the inner strap 18, as transferred to the hooked catches by the windlass 74. In accordance with embodiments of the present disclosure, and by way of example and not limitation, the hooked catches 82 may be formed of a KYDEX® thermoplastic material or molded plastic that may be integrally formed with, or otherwise connected to the base member 62. The preferred use of two opposing catches 82 allows the user to rotate the windlass 74 in either direction, with one of the two catches 82 always able to prevent the windlass 74 from unwinding. However, it is to be understood that the use of a single hooked catch 82 may be used and is within the scope of the present disclosure. For a single hooked catch 82, the user must rotate the windlass in the proper direction to allow the tension in the inner strap 18 to be resisted by the single hooked catch 82 once winding of the windlass and tensioning of the inner strap 18 is completed.

In accordance with embodiments of the present disclosure, the securing mechanism 26 may comprise a securing strap positioned transversely to a longitudinal axis L-L of the outer sleeve 14. As for example, a transversely oriented strap having hook and loop fastening portions, or an elastic band engaging a hook or button may be provided to secure the windlass 74 in its wound position.

In yet another possible alternative, a retainer 86, for example, a transversely oriented strap 86 may be used in combination with the hooked catches 82. Such a combination of structures allows the user to secure the windlass 74 and move about (or be moved by another person) with less concern of the windlass 74 dislodging from the hooked catches 82 and unwinding. In accordance with embodiments of the present disclosure, for hooked catches 82 used in combination with a transversely oriented strap 86, the outer surface of the hooked catches may comprise a hook or loop material, and a surface of the strap 86 may comprise a complementary hook or loop material to interlock with the material on the hooked catches 82. According to some examples, the catches 82 may be implemented as one of a hook and loop structure, magnets, and buttons. It may be appreciated that other materials may be used.

As shown in FIG. 2, according to one or more embodiments, the buckle 38 may include an area 53 having an indication thereon, for example, imprinted, etched, labeled, or otherwise providing an indication that assists in the application of the tourniquet to an injured person by the user. Further, windlass 74 may include areas 35, 37, and 39, having an indication thereon, for example, imprinted, etched, labeled, or otherwise providing an indication, such as numbers, words, images, etc., that assists in the application of the tourniquet to an injured person by the user. Further, an area 33 and 4 may have an indication thereon, for example, imprinted, etched, labeled, or otherwise providing an indication that assists in the application of the tourniquet to an injured person. Still further, strap 86 may include a timer section 43. The timer section may be set when the tourniquet is applied in order to indicate to medical personnel how long the tourniquet has been applied to the injured person. The timer section 43 may include a display 49, an activation mechanism 45 such as a timer starter to start the timer, for example, a start button, a timer reset 47 and circuitry for operating the timer. The timer reset may be implemented as a button that is depressed into the strap 86 so that it is difficult to reset. This may prevent the timer from accidentally being reset. Strap 86 may further include area 51 having an indication thereon, for example, imprinted, etched, labeled, or otherwise providing an indication that assists in the application of the tourniquet to an injured person by the user. The indications may provide instructions to a user as to how to properly apply the tourniquet to an injured person.

It may be appreciated that according to some embodiments, the timer section may not include a timer reset 47.

According to some embodiments, the timer section may be configured such that the activation mechanism may be activated automatically when one or more portions of the tourniquet are engaged or disengaged. For example, when the outer packaging is removed, a plastic device that may be inserted during manufacturing may be removed such that a circuit may be completed for the timer section thereby allowing the timer to start. For another example, when the tourniquet is applied to an injured person and the strap is looped through the buckle and secured back to itself, a circuit may be completed thereby automatically activating the activation mechanism of the timer.

According to one or more embodiments, strap 86 may further include an audio section 81. Audio section 81 may include a speaker 77, such as a microspeaker, an activation mechanism 79, for example, a pushbutton, circuitry and memory for storing a pre-recorded message and operating the speaker section. The audio section 81 may further include circuitry (not shown) for storing a pre-recorded message such that when the pushbutton 79 is depressed, instructions for how to apply the tourniquet may play through the speaker 77. It may be appreciated that the circuitry for operating the timer section 43 and the circuitry for operating the speaker section may be integrated or may be separately provided. Further, according to some embodiments, the activation mechanism 79 and the timer starter 45 may be implemented as one activation mechanism.

According to some embodiments, two speakers, such as microspeakers may be used. One speaker may be a self-contained speaker including a battery and may be configured such that when an outer packaging of the tourniquet is removed, the pre-recorded set of instructions automatically plays one or more predetermined times. The user may further replay the prerecorded set of instructions by the activation mechanism. For example, when the outer packaging is removed, a plastic device that may be inserted during manufacturing may be removed such that a circuit may be completed for the audio section thereby allowing the prerecorded instructions to start.

FIG. 25 depicts a front and back view of a tourniquet according to some embodiments of the present disclosure. As shown in FIG. 25, instructions on how to apply the tourniquet to an injured person may be automatically played upon removing packaging materials.

According to one or more embodiments, the audio section 81 may include two speakers, such as microspeakers may be used. One speaker may be a self-contained speaker including a battery and may be configured such that when an outer packaging of the tourniquet is removed, the pre-recorded set of instructions automatically plays one or more predetermined times. The user may further replay the prerecorded set of instructions by the activation mechanism. Further, the tourniquet may include circuitry for a physiologic tracking device. When the outer packing of the tourniquet is removed, the tracking device may automatically start similarly to how the audio automatically starts. The physiological tracker may indicate pulse and/or other bio markers as programmed and may provide indications via light emitting diodes, may store the tracked biomarkers, may transmit the biomarkers to a remove device, such as a server, a mobile application, etc. The speaker(s) and the circuit boards may be provided between the hook and loop layer and the material of the strap.

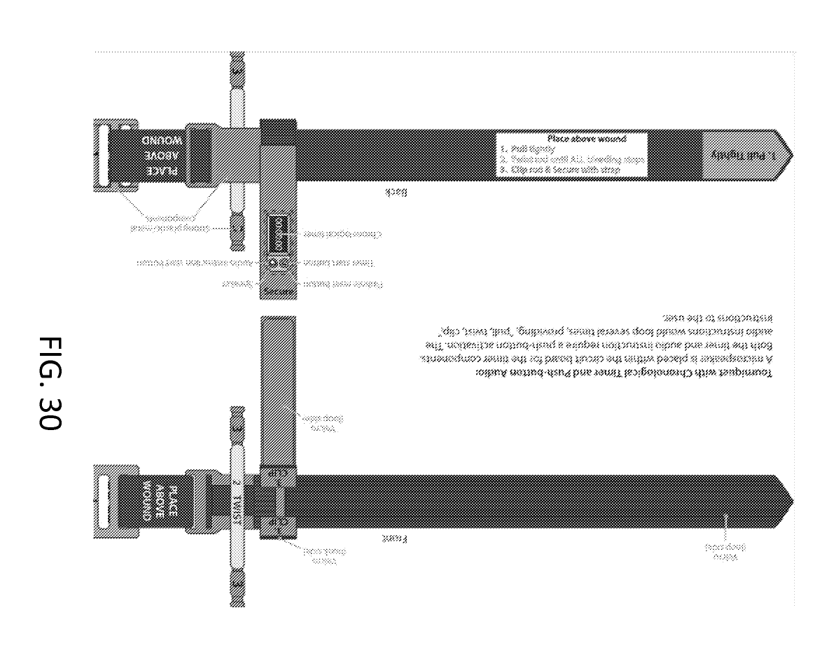

FIG. 28 depicts a front and back view of a tourniquet according to some embodiments where the timer section, speaker section, and physiologic tracking device automatically starts once the packaging is removed.

According to some embodiments, the audio section and/or the timer section may be configured such that when the windlass rod is secured in the catch, a circuit is completed thereby activating the activation mechanism such that the timer automatically starts.

FIG. 27 depicts a front and back view of a tourniquet according to some embodiments where the timer section automatically starts once the windlass rode is secured into the catch.

According to one or more embodiments, the audio section 81 may include two speakers, such as microspeakers may be used. One speaker may be a self-contained speaker including a battery and may be configured such that when an outer packaging of the tourniquet is removed, the pre-recorded set of instructions automatically plays one or more predetermined times. The user may further replay the prerecorded set of instructions by the activation mechanism. Further, a plastic barrier may be inserted during the manufacturing process on the windlass securing strap such that when the plastic is removed from the windlass, the timer automatically starts when the strap is secured.

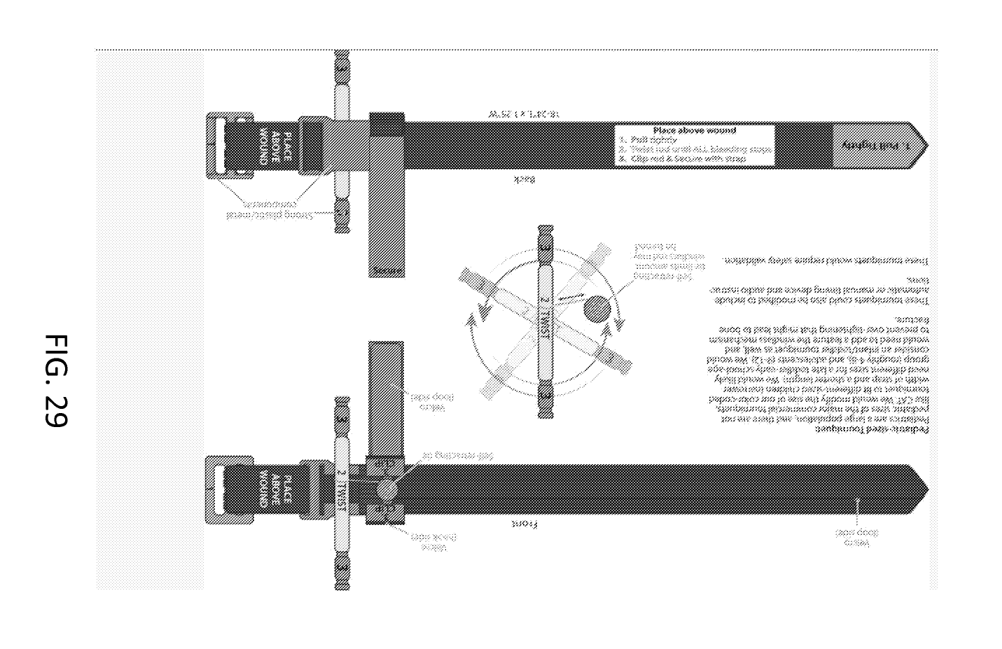

FIG. 26 depicts a front and back view of a tourniquet according to some embodiments of the present disclosure. As shown in FIG. 25, instructions on how to apply the tourniquet to an injured person may be automatically played upon removing packaging materials.

According to one or more embodiments, as depicted in FIG. 29, the size of the tourniquets as described herein may be reduced to pediatric sizes, where the width of the strap may be narrowed and shortened. For example, the width and length may be reduced for a size to fit late toddler/school age (ages about 4 to 8 years of age), and adolescents (ages of about 9 to 12 years of age). The length and width of the strap may further be reduced in order to accommodate infants where the windless may be removed in order to prevent over-tightening that might lead to bone fracture.

According to one or more embodiments, as depicted in FIG. 30, a front and back view of a tourniquet is depicted including a timer section and an audio section having a timer activation mechanism and an audio instruction activation mechanism.

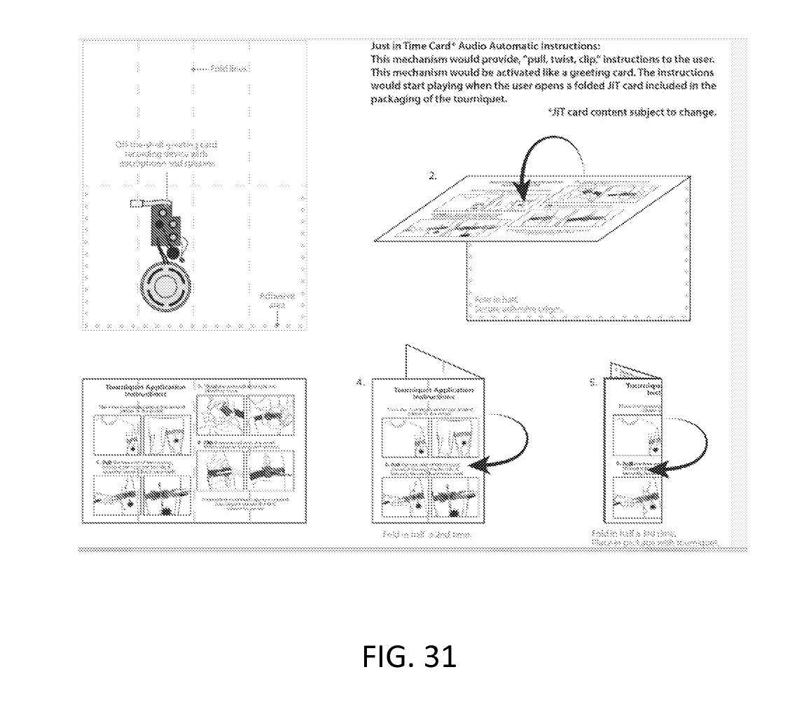

According to one or more embodiments, as depicted in FIG. 31, a set of printed instructions is provided including an audio section. The audio section may include an activation mechanism that activates automatically upon opening of the printed set of instructions. Upon activation, audio instructions may be played through speakers thereby providing audio instructions to a user on how to apply the tourniquet to an injured person.

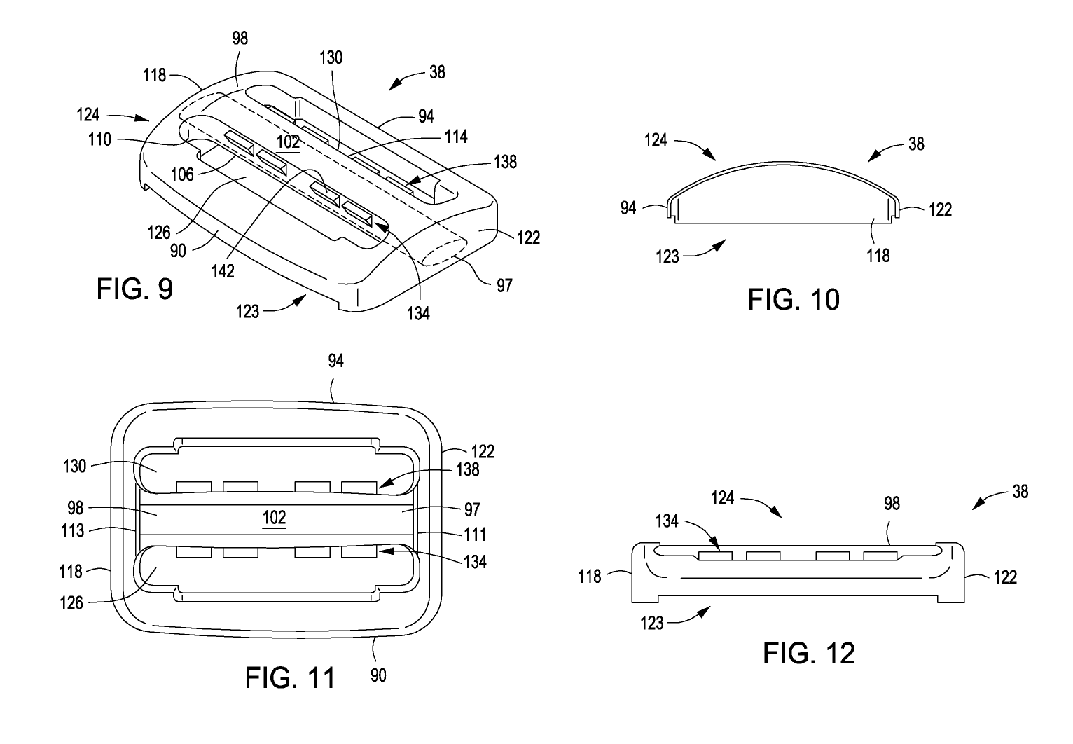

With reference to FIGS. 9-12, an embodiment of a buckle 38 is shown for use in the above-described embodiments of the present disclosure. As previously described, the first end 30 of the outer sleeve 14 is looped through the buckle 38 and pulled tight around the appendage to allow the outer sleeve 14 to circumferentially surround or encircle the appendage. More specifically, the buckle 38 includes a first lateral side 90, a second lateral side 94, and an intermediate bar 98 generally parallel to and located between the first lateral side 90 and the second lateral side 94. The intermediate bar 98 includes a top surface 102, a bottom surface 106, and a first sidewall 110 and a second sidewall 114 located between the top surface 102 and bottom surface 106. A first end 118 and a second end 122 of the buckle 38 interconnect the first lateral side 90, second lateral side 94, and intermediate bar 98. Located between the first lateral side 90 and the intermediate bar 98 is a first port 126, and formed between the second lateral side 94 and the intermediate bar 98 is a second port 130. Each port provides a route or pathway for the looping outer sleeve 14 through the buckle 38 during tightening and loosening of the outer sleeve around the appendage. Buckle 38 further includes a barrier bar 97. Barrier bar 97 includes a top surface 99, a bottom surface 101, a first side wall 103, and a second sidewall 105, as depicted in FIG. 14. A first end 111 and a second end 113 of barrier bar 97 of buckle 38 interconnect the first lateral side 90, second lateral side 94, and barrier bar 97. Located between the first lateral side 90 and the intermediate bar 98 and barrier bar 97 is a first port 126, and formed between the second lateral side 94 and the intermediate bar 98 and barrier bar 97 is a second port 130. Formed between the intermediate bar 98 and the barrier bar 97 is a third port 121. Each port provides a route or pathway for the looping outer sleeve 14 through the buckle 38 during tightening and loosening of the outer sleeve around the appendage.

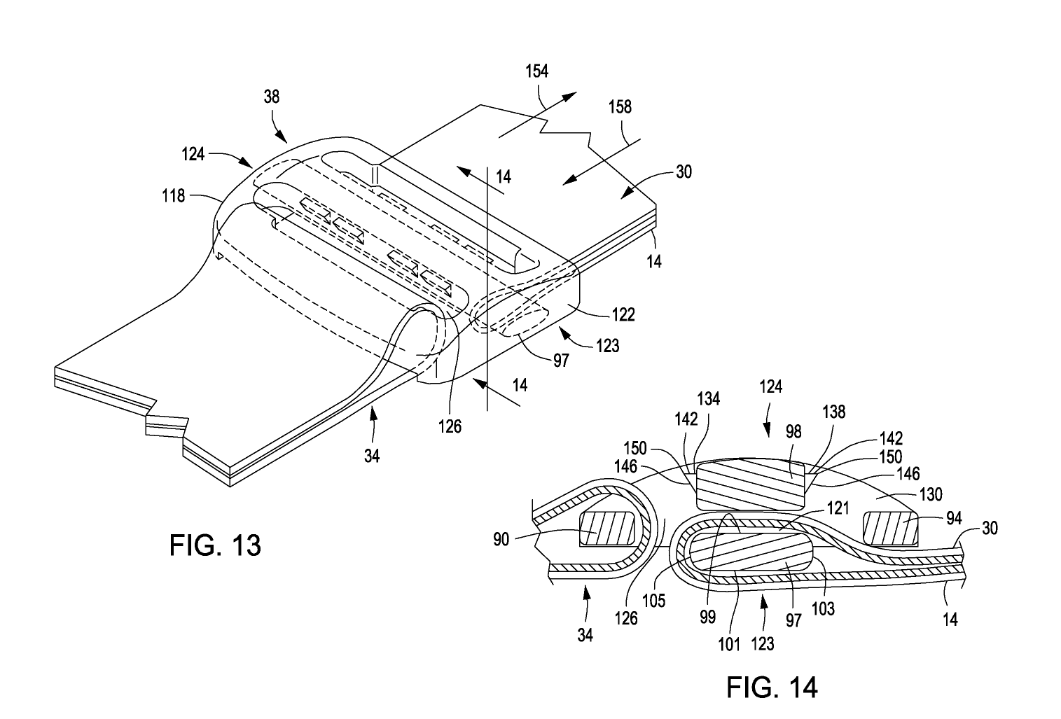

Referring now to FIGS. 9, 13 and 14, the first port 126 includes a first tooth set 134 mounted to the first sidewall 110, and the second port 130 includes a second tooth set 138 mounted to the second sidewall 114 of the intermediate bar 98 for inhibiting movement of the outer sleeve 14 with respect to the buckle 38. Each tooth set 134 and 138 comprises at least one tooth. With reference to FIG. 14, each tooth includes a top surface 142, an inclined surface 146, and an edge or projection 150 therebetween. During operation and with respect to FIG. 13 and FIG. 14, the first end 30 of the outer sleeve 14 is fed through the third port 121 from the bottom 123 over the top of and around barrier bar 97, to the bottom 123 of the buckle 38. After the first end 30 has exited the third port 121 and been pulled to a desired tightness around the appendage, the hook and loop fastener on the bottom surface 50 of the first end 30 of the outer sleeve 14 is mated with the hook and loop fastener on the bottom surface 50 of the remainder of the outer sleeve 14 in order to secure the outer sleeve 14 around the appendage.

Referring still to FIGS. 13 and 14, a more detailed view illustrating the interaction between the first tooth set 134 and second tooth set 138 and the outer sleeve 14 is shown. More specifically, once the first end 30 of the outer sleeve 14 has been fed through the third port 121, the projections 150 of the first tool set 134 and second tooth set 138 of the first port 126 and second port 130 engage with the hooks and loops on the outer surface 50 of the outer sleeve 14.

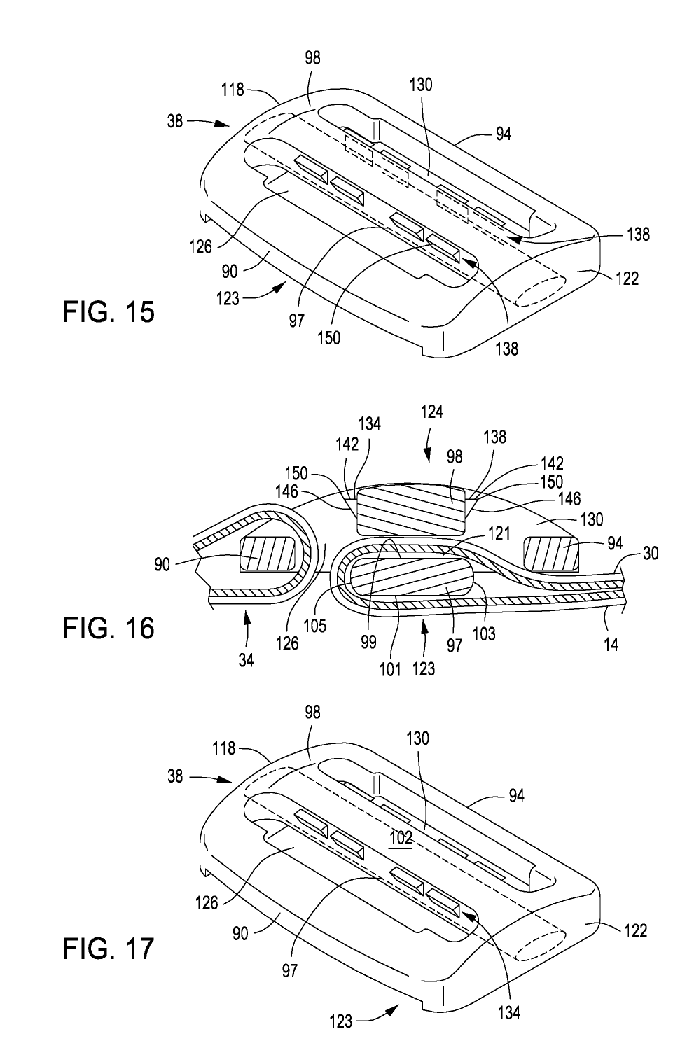

With respect to FIGS. 15 and 16, a variation of the buckle 38 is illustrated. More specifically, the second tooth set 138 is inverted relative to the first tooth set 134.

Referring now to FIG. 17, and in accordance with another embodiment of the buckle 38, only a first tooth set 134 is provided on the buckle.

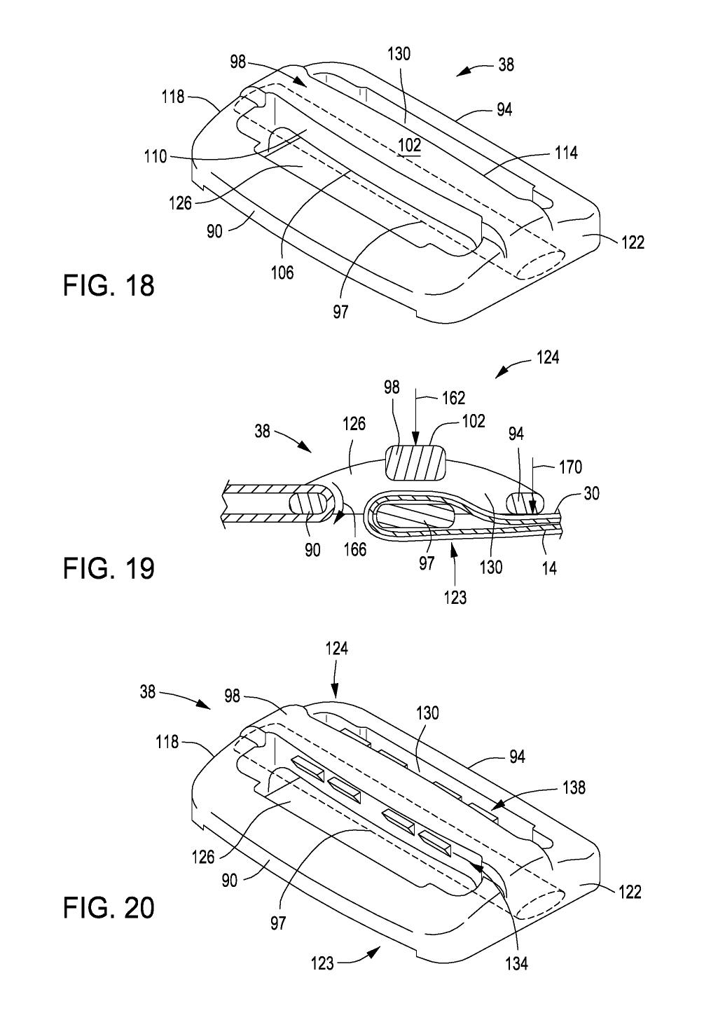

With reference now to FIGS. 18 and 19, another embodiment of the buckle 38 is shown. Similar to previous embodiments, the buckle 38 includes first lateral side 90 and second lateral side 94, first end 118 and second end 122, first port 126 and second port 130, and an intermediate bar 98. The intermediate bar 98 includes a top surface 102, a bottom surface 106, and first sidewall 110 and second sidewall 114 located between the top surface 102 and bottom surface 106. However, as best seen in FIG. 19, one can see that the top surface 102 of the intermediate bar 98 is elevated relative to the first end 118 and second end 122 of the buckle 38. Buckle 38 further includes barrier bar 97. Barrier bar 97 includes a top surface 99, a bottom surface 101, a first side wall 103, and a second sidewall 105. A first end 111 and a second end 113 of barrier bar 97 of buckle 38 interconnect the first lateral side 90, second lateral side 94, and barrier bar 97. Located between the first lateral side 90 and the intermediate bar 98 and barrier bar 97 is a first port 126, and formed between the second lateral side 94 and the intermediate bar 98 and barrier bar 97 is a second port 130. Formed between the intermediate bar 98 and the barrier bar 97 is a third port 121. Each port provides a route or pathway for the looping outer sleeve 14 through the buckle 38 during tightening and loosening of the outer sleeve around the appendage.

In operation, and with reference to FIG. 19, the outer sleeve 14 is routed through the buckle 38 as generally shown by arrow A, wherein the first end 30 of the outer sleeve 14 is fed through the second port 130 from the bottom 123 to the top 124 of the buckle 38. After the first end 30 has exited the second port 130 and been pulled to a desired tightness around the appendage, the hook and loop fastener on the outer surface 50 of the first end 30 of the outer sleeve 14 is mated with the hook and loop fastener on the outer surface 50 of the remainder of the outer sleeve 14 in order to mount the outer sleeve 14 around the appendage. Further, as the first end 30 of the outer sleeve 14 is being pulled from the second port 130, an input load 162 is applied generally perpendicularly to the top surface 102 of the elevated intermediate bar 98. The input load 162 causes the buckle to pivot per arrow 166 around the first lateral side 90 of the buckle 38, wherein the first lateral side 90 essentially acts as a fulcrum. As a result, an output load 170 is produced at the second lateral side 94 of the buckle 38 which forces the second lateral side 94 against the outer sleeve 14, thereby interlocking the outer surface 50 of the outer sleeve 14. It will be appreciated that as more of the outer sleeve 14 is pulled through the second port 130, the output load 170 applied to the outer sleeve 14 increases providing for a more sturdy arrangement.

Referring now to FIG. 20, another embodiment of the buckle 38 of the present disclosure is shown. More specifically, the buckle 38 includes an elevated intermediate bar 98 along with first tooth set 134 and second tooth set 138 situated adjacent the first port 126 and second port 130.

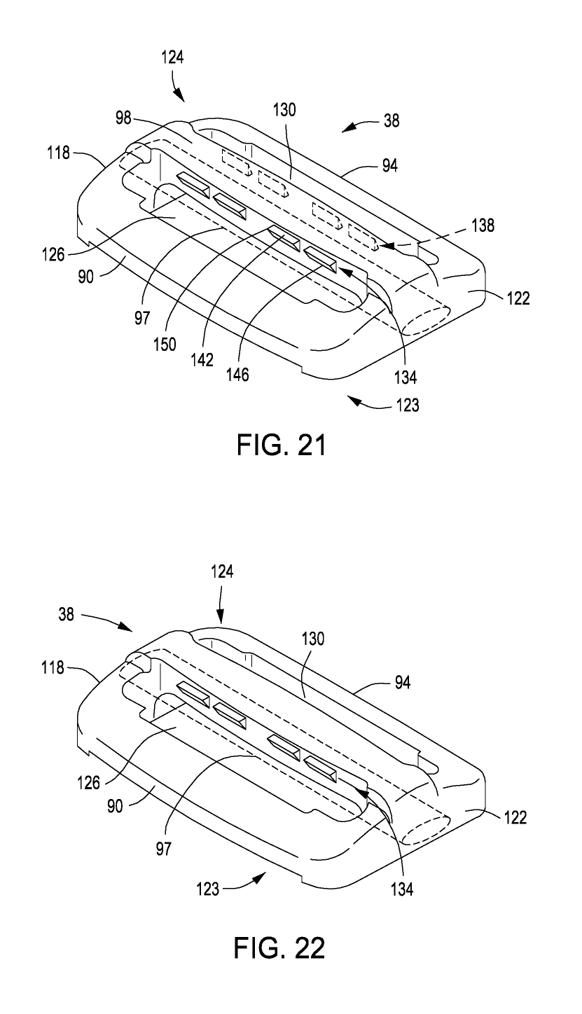

With reference now to FIG. 21, a buckle 38 is provided similar to the buckle 38 of FIG. 20, but with the second tooth set 138 inverted. Such an arrangement allows the outer sleeve 14

With reference now to FIG. 22, a buckle 38 is illustrated with an elevated intermediate bar 98 and only a first tooth set 134. As previously described, such an arrangement advantageously simplifies manufacturing of the buckle in addition to facilitating tightening and loosening movement of the outer sleeve 14 within the buckle 38.

Those of ordinary skill in the art will appreciate that other variations of the buckle 38 are within the scope of the present disclosure. For instance, while the first tooth set 134 of FIG. 22 is illustrated as being situated in the first port 126, ordinary artisans will appreciate that the first tooth set 134 can be situated in the second port 130, if desired. Additionally, it is contemplated that any of the aforementioned tooth sets could be inverted or not inverted so as to provide a desired amount of drag during loosening or tightening movement of the outer sleeve 14. Relevant considerations in determining which features to incorporate into a particular buckle 38 may include ease and cost of manufacturing, ease of assembly of the outer sleeve 14 and the buckle 38, the familiarity of the operators with the buckle 38, desired amount of drag applied to the outer sleeve 14, etc.

Further, while four teeth are shown in each tooth set in some of the above embodiments, it is contemplated that fewer teeth (including only a single tooth) may be provided to facilitate manufacturing, to provide for fewer sharp edges or for other reasons that an operator may desire. Additionally, more than four teeth may be provided to increase the gripping ability of the tooth set. Further, while each of the teeth is shown to be of a triangular profile, teeth of other shapes, are within the scope of the present disclosure such as bulbous-shaped teeth, teeth having only a single sharp point, teeth having two inclined surfaces, etc. Also, the angle of any inclined surfaces can be modified in order to provide more or less drag on the outer sleeve.

While the buckle of the present disclosure is preferably constructed of a polymer such as plastic or rubber, it is also contemplated that the buckle could be constructed of other materials such as metals, composites, etc.

It may be further appreciated that any of the embodiments described herein may be implemented without any teeth at all.

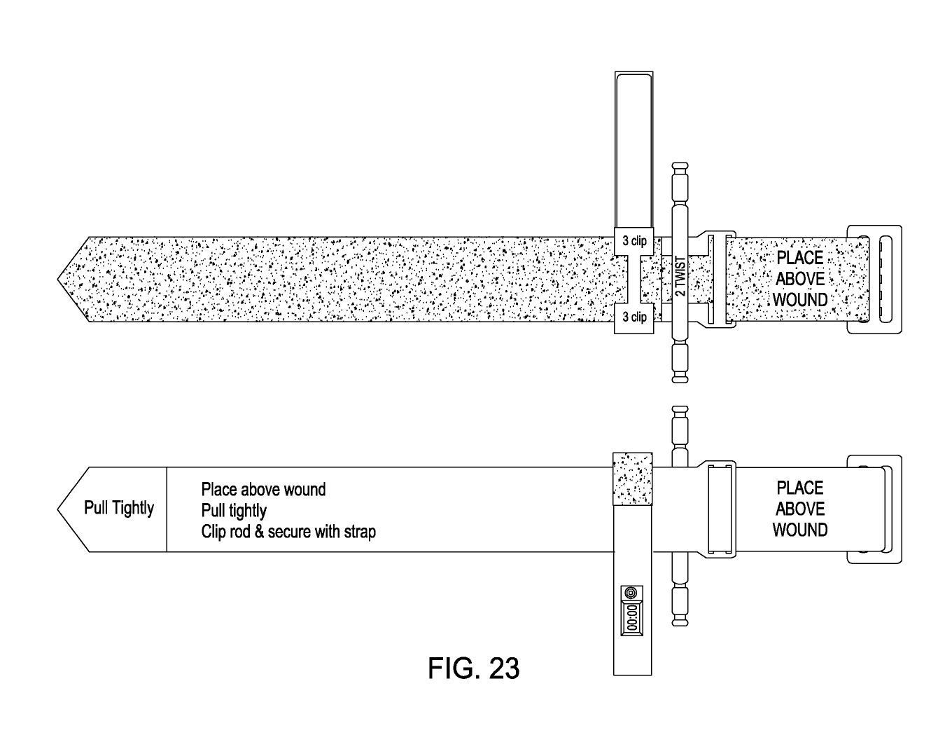

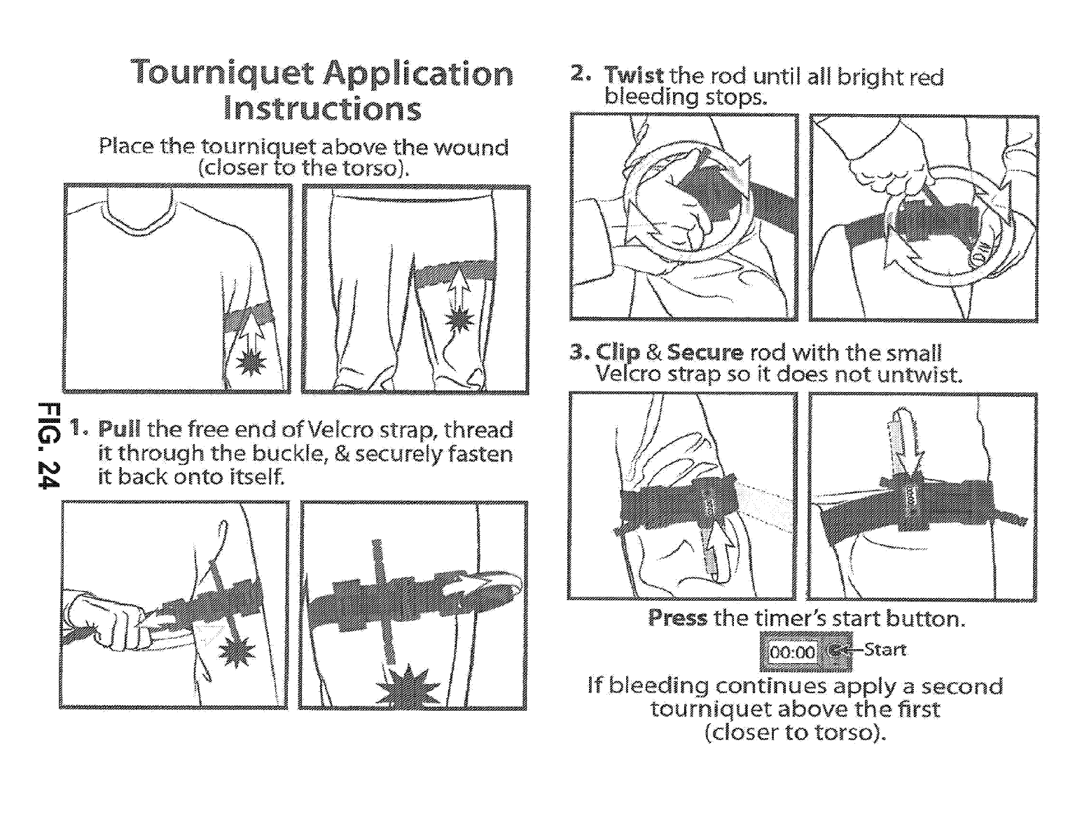

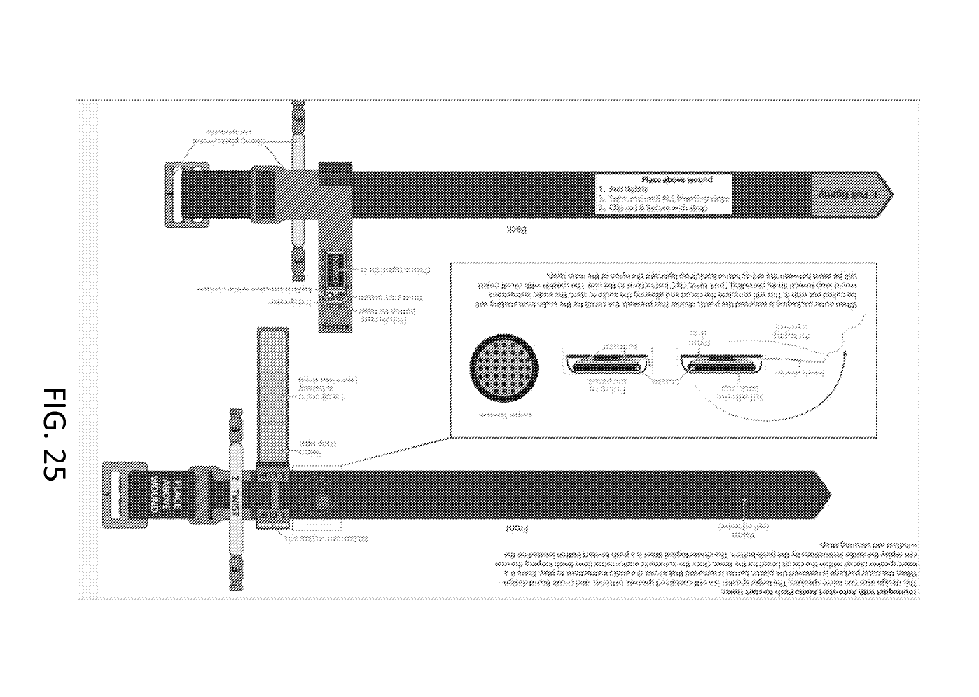

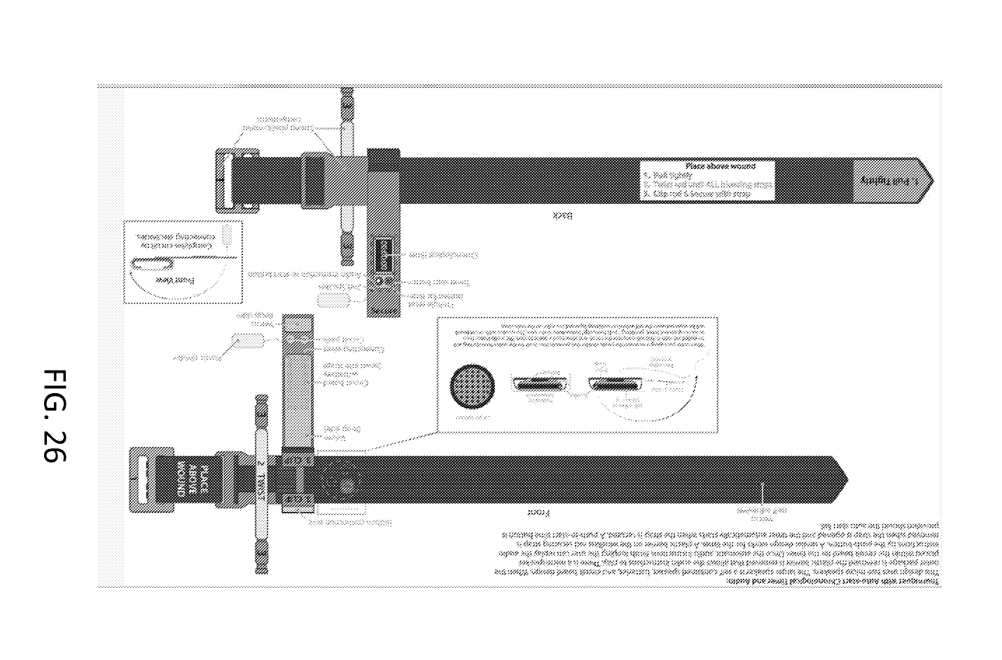

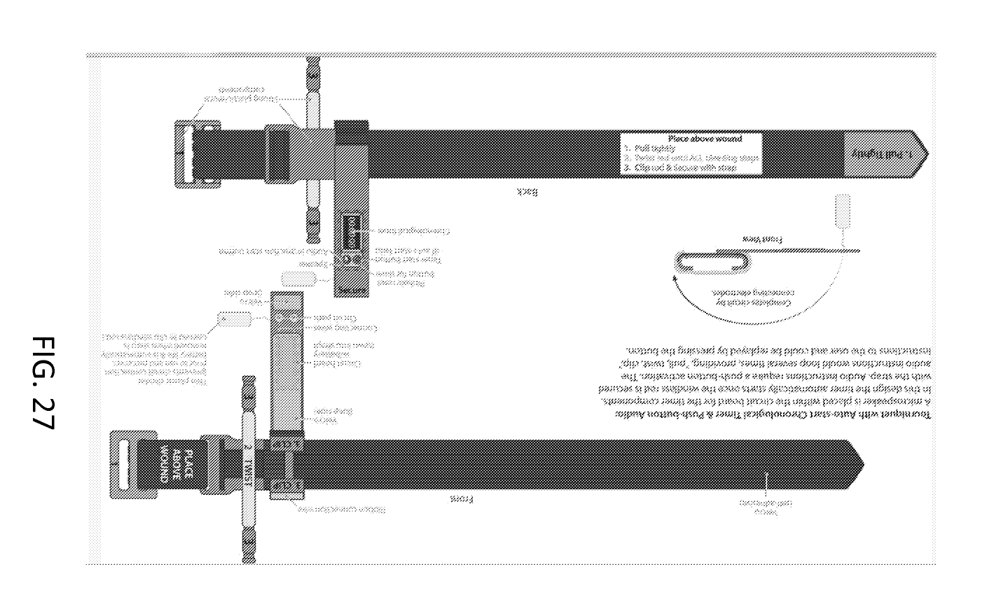

FIG. 23 depict a tourniquet in accordance with one or more embodiments, including areas for including printed instructions regarding how to the properly apply the tourniquet to an injured person. The different areas may be color coded. For example, the windlass rod includes a central portion and a peripheral portion and wherein the central portion is of a first color. The slide buckle may be of a second color and the base member, the retainer, and the catch may be a third color and each of the first color, the second color, and the third color is different. The peripheral portion of the windlass may be the same color as the base member, the retainer, and the clip. At least one instruction of the set of instructions relates to operation of the slide buckle, the central portion or peripheral portion of the windlass rod, or the retainer, and the at least one instruction of the set of instructions is provided in the same color of the slide buckle, the central or peripheral portion of the windlass rod, or the retainer that the at least one instruction of the set instructions corresponds to. By color coding the instructions, and the portions of the tourniquet to which the instruction applies to, the user may easily understand how to properly apply the tourniquet to an injured person, even if the user is not experienced medical personnel.

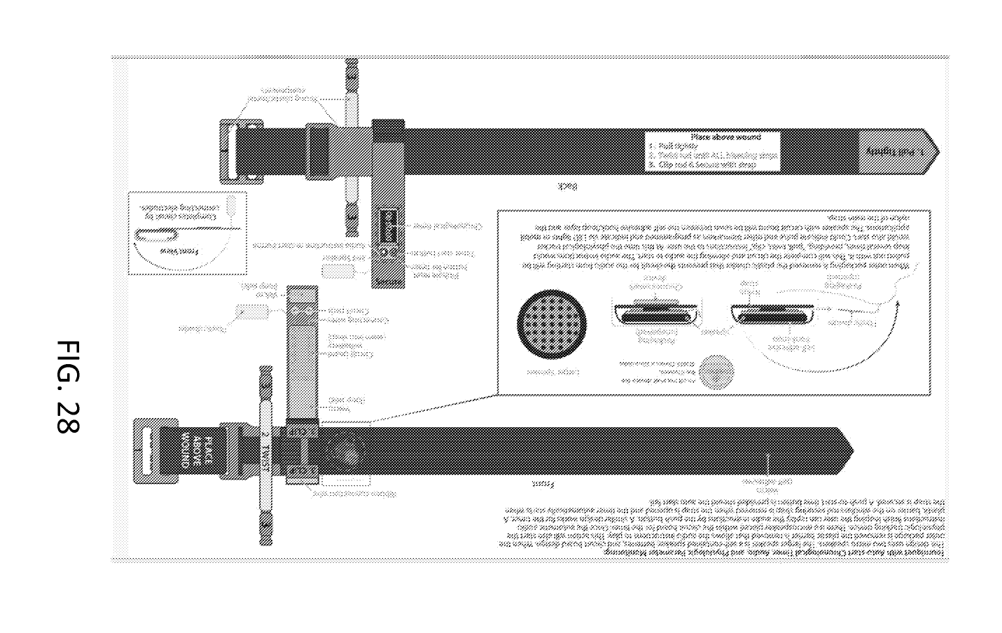

FIG. 24 depicts an example set of printed instructions that may be available to the user of the tourniquet. The instructions may be printed in black and white or may be printed in color such that the color of the instructions corresponds to those areas of the tourniquet that are of the same color. This may further assist the user in instructing the user how to apply the tourniquet properly.

The present disclosure has application for use in emergency medical situations for people. In addition, the disclosure also has application for use in veterinary medicine to apply a tourniquet to a body part or limb of an animal.

The present disclosure, in various embodiments, includes components, methods, processes, systems and/or apparatus substantially as depicted and described herein, including various embodiments, subcombinations, and subsets thereof. Those of skill in the art will understand how to make and use the present disclosure after understanding the present disclosure. The present disclosure, in various embodiments, includes providing devices and processes in the absence of items not depicted and/or described herein or in various embodiments hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover though the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A slide buckle, comprising:
a frame;
an intermediate bar integrally connected at two opposite sides of the frame and defining two ports in the frame; and
a barrier bar integrally connected at the two opposite sides of the frame and spaced apart from the intermediate bar forming a third port between the intermediate bar and the barrier bar.

2. The slide buckle of claim 1, wherein the bar includes a plurality of protrusions extending into at least one of the two slots, wherein the plurality of protrusions prevent slipping of straps threaded in the respective two ports.

3. A tourniquet, comprising:
the slide buckle of claim 1;
a base member comprising:
a base member frame;
a base member intermediate bar defining two ports in the base member frame, wherein a portion of a second strap is threaded through one of the two ports in the base member frame and another portion of the second strap is threaded through one of the two ports in the frame of the slide buckle and the third port of the slide buckle; and
an extension portion of the base member; and
the first strap;
wherein the first strap has a first end connected to the extension portion of the base member.

4. The tourniquet of claim 3, further comprising:
a retainer, comprising:
a first portion having a catch for securing a windlass rod, the catch having at least one top portion, where a surface of the top portion includes a first coupling member; and
a second portion having a third strap, where the third strap includes a second coupling member.

5. The tourniquet of claim 3, wherein the first and second coupling members made of material including one of a hook and loop structure, magnets, and buttons.

6. The tourniquet of claim 3, wherein the tourniquet includes at least one area including a set of instructions for applying the tourniquet.

7. The tourniquet of claim 4, wherein the third strap includes a timing device.

8. The tourniquet of claim 7, wherein the timing device includes a reset feature to reset a timing of the timing device, the reset feature being configured to be depressed into a surface of the timing device.

9. The tourniquet of claim 4, wherein the windlass rod is operatively associated with a fourth strap and configured to engage with the catch of the retainer.

10. The tourniquet of claim 4, wherein the windlass rod includes a central portion and a peripheral portion and wherein the central portion is of a first color.

11. The tourniquet of claim 10, wherein the slide buckle is a second color and the base member, the retainer, and the catch are a third color and each of the first color, the second color, and the third color is different.

12. The tourniquet of claim 10, wherein the peripheral portion of the windlass is the same color as the base member, the retainer, and the clip.

13. The tourniquet of claim 12, wherein the tourniquet has imprinted thereon a set of instructions, and at least one instruction of the set of instructions relates to operation of the slide buckle, the central portion or peripheral portion of the windlass rod, or the retainer, and the at least one instruction of the set of instructions is provided in the same color of the slide buckle, the central or peripheral portion of the windlass rod, or the retainer that the at least one instruction of the set instructions corresponds to.

14. The tourniquet of claim 3, further comprising:
an audio section having an activation mechanism; and
a speaker;
wherein the audio section is configured to play pre-recorded instructions for using the tourniquet.

15. The tourniquet of claim 3, further comprising:
a physiological tracker for collecting and storing at least one biomarker.

16. The tourniquet of claim 3, further comprising:
a timer section comprising:
an activation mechanism; and
a display.

17. The tourniquet of claim 16, wherein the activation mechanism is automatically activated upon application of the tourniquet to an injured person.
